(19) United States
(12) Patent Application Publication
(10) Pub. No.: US 2021/0340137 A1
(43) Pub. Date: Nov. 4, 2021

(54) LIGHT OUT-COUPLING MATERIAL, MANUFACTURING METHOD THEREOF, AND ELECTROLUMINESCENT DEVICE

(71) Applicant: Wuhan China Star Optoelectronics Semiconductor Display Technology Co., Ltd., Wuhan, Hubei (CN)

(72) Inventors: Xu Wang, Wuhan (CN); Jiajia Luo, Wuhan, Hubei (CN)

(73) Assignee: Wuhan China Star Optoelectronics Semiconductor Display Technology Co., Ltd., Wuhan, Hubei (CN)

(21) Appl. No.: 16/625,322

(22) PCT Filed: Oct. 12, 2019
(86) PCT No.: PCT/CN2019/110698

§ 371 Date: Dec. 20, 2019

(30) Foreign Application Priority Data
Jul. 23, 2019 (CN) 201910663913.4

(51) Int. Cl. C07D 471/04; H01L 51/00

(52) U.S. Cl. CPC C07D 471/04; H01L 51/0052; H01L 51/5016; H01L 51/0094; H01L 51/0072

(57) ABSTRACT

A light out-coupling material, a manufacturing method thereof, and an electroluminescent device are provided. A long-chain light out-coupling material is designed, specifically by attaching a long-chain naphthalene to phenanthroline as a bridging center and attaching other groups having a narrow absorption band to both ends of the bridging center to undergo arrangement, so that the light out-coupling material can be arranged in a flat orientation during an evaporation process. Therefore, the light out-coupling material has a high refractive index. At last, the light out-coupling material of the target compound is applied to a light out-coupling layer of the electroluminescent device, thereby achieving high efficiency. Moreover, a thickness of the light out-coupling layer of the electroluminescent device is reduced from 85 nm to 65 nm, which effectively saves time and capital costs.

### FIELD OF INVENTION

The present disclosure relates to a field of display technologies, and more particularly to a light out-coupling material, a manufacturing method thereof, and an electroluminescence device.

### BACKGROUND OF INVENTION

Organic light-emitting diodes (OLEDs) possess many advantages, such as self-luminosity that does not require a backlight, high luminous efficiency, wide viewing angles, fast response speeds, wide temperature adaptation range, simple manufacturing and processing technique, low driving voltages, low energy consumption, lighter and thinner, flexible display, etc. OLEDs possess huge application prospects which have attracted attention of many researchers.

### Technical Problems

For currently used top-emitting device structure, microcavity effect can greatly increase device efficiency, narrow spectrum, and broaden a color gamut. In the device structure, light out-coupling layers (CPL) play a huge role. High refractive index (N) material can not only enhance device efficiency, but also reduce a thickness of the material, thereby accomplishing purpose of saving material and reducing cost. General CPL material has a low refractive index and thus a thickness of the general CPL material used in the evaporation process is greater than 85 nm.

### SUMMARY OF INVENTION

In order to solve the above technical problems, the present application design a long-chain light out-coupling material so that the light out-coupling material can be arranged in a flat orientation during an evaporation process. Therefore, the light out-coupling material has a high refractive index value.

A technical solution to solve the above problems is that the present application provides a light out-coupling material comprising the following general structural formula:

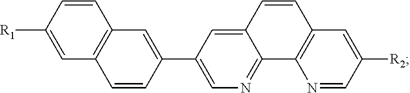

wherein a group R₁ and a group R₂ each comprises one of an alkyl group, an alkoxy group, or an aromatic group.

Furthermore, the group R₁ comprises one of the following structural formulas:

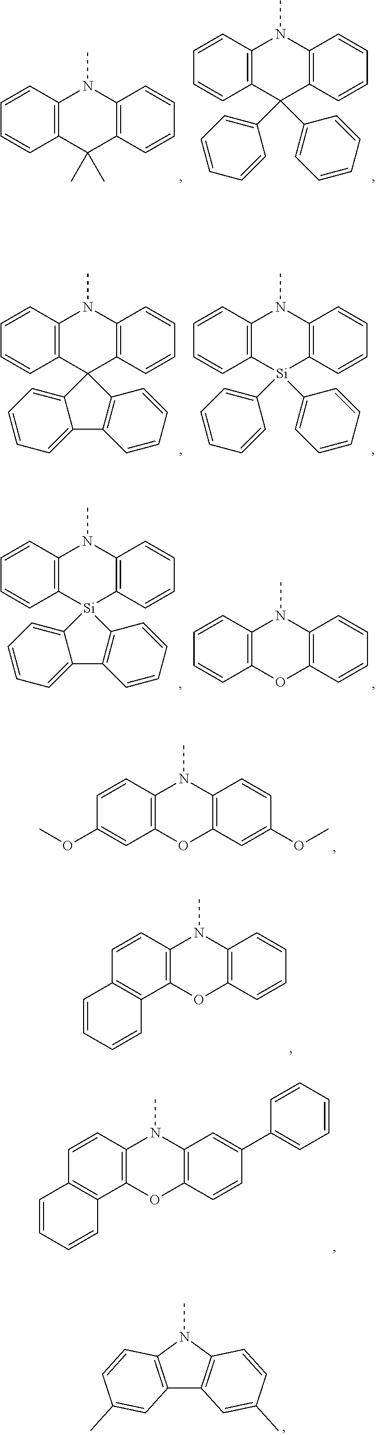

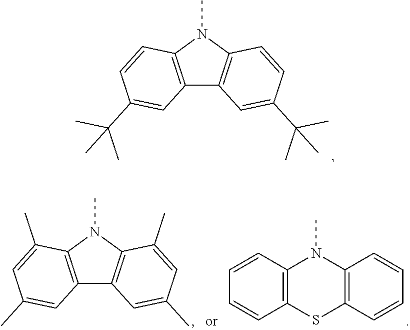

Furthermore, the group R₂ comprises one of the following structural formulas:

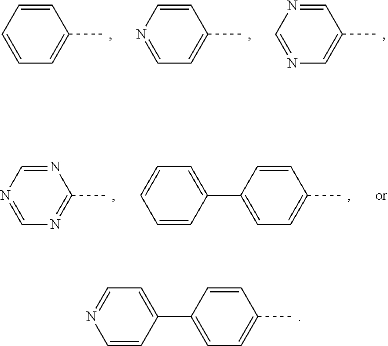

The present disclosure also provides a manufacturing method of the light out-coupling material, wherein the manufacturing method comprises the following steps: preparing an intermediate having the group R₂, a naphthalene, and a phenanthroline; adding the intermediate and a first raw material having the group R1, a catalyst, and sodium t-butoxide into a three-neck flask, and performing gas evacuation and refilling by argon gas; adding anhydrous toluene into the reaction vessel to undergo a reaction at a temperature ranging from 110° C. to 130° C. for 24 h, and cooling to room temperature to obtain a first mixed solution; introducing the first mixed solution into 180 to 220 ml of ice water, and extracting with dichloromethane several times to obtain an extract solution; and drying the extract solution with anhydrous sodium sulfate, filtering and spin-drying the extract solution, then subjecting the extract solution to column chromatography using 200 to 300 mesh of silica gel, and being rinsed with an eluent to obtain the light out-coupling material.

Furthermore, the step of preparing the intermediate specifically comprises the following steps: adding 3-(6-Bromonaphthalen-2-yl)-8-iodo-1,10-phenanthroline, phenylboronic acid, and a catalyst into a Schlenk bottle, and introducing argon gas into the Schlenk bottle; adding deoxygenated toluene, deoxygenated ethanol, and deoxygenated water into the Schlenk bottle, heating the Schlenk bottle under argon protection, and undergoing a reaction at a temperature ranging from 70° C. to 90° C. for 24 h to obtain a second mixture solution; extracting the second mixed solution with dichloromethane several times to obtain a first extract solution; and drying the first extract solution with anhydrous sodium sulfate, filtering and spin-drying the first extract solution, then subjecting the first extract solution to column chromatography using 200 to 300 mesh of silica gel, and being rinsed with an eluent to obtain the intermediate.

Furthermore, the first raw material comprises phenothiazine, 9,10-dihydro-9,9-dimethylacridine, and 3,6-dimethylcarbazole; a molar ratio of the first raw material to the intermediate is 5:8 to 5:6; the catalyst comprises palladium acetate and tri-tert-butylphosphine tetrafluoroborate; and a molar ratio of the palladium acetate to the tri-tert-butylphosphine tetrafluoroborate is 1:5 to 1:3.

Furthermore, a molar ratio of the 3-(6-bromonaphthalen-2-yl)-8-iodo-1,10-phenanthroline to the phenylboronic acid is from 10:9 to 10:5.

The present disclosure also provides also provides an electroluminescent device comprising the light out-coupling material.

Furthermore, the electroluminescent device comprises: a first electrode; a light-emitting functional layer disposed on the first electrode; a second electrode disposed on the light-emitting functional layer; and a light out-coupling layer disposed on the second electrode, wherein a material used for the light out-coupling layer comprises the light out-coupling material.

Furthermore, the luminescent functional layer comprises: a hole injection layer disposed on the first electrode; a hole transport layer disposed on a side of the first electrode of the hole injection layer; an electron blocking layer disposed on a side of the hole transport layer that is away from the hole injection layer; a light-emitting layer disposed on a side of the electron blocking layer that is away from the hole transport layer; a hole blocking layer disposed on a side of the light-emitting layer that is away from the electron blocking layer; an electron transport layer disposed on a side of the hole blocking layer that is away from the light-emitting layer; and an electron injection layer disposed on a side of the electron transport layer that is away from the hole blocking layer.

### Beneficial Effects

The present disclosure provides a light out-coupling material, a manufacturing method thereof, and an electroluminescent device. A long-chain light out-coupling material is designed, specifically by attaching a long-chain naphthalene to phenanthroline as a bridging center and attaching other groups having a narrow absorption band to both ends of the bridging center to undergo arrangement, so that the light out-coupling material can be arranged in a flat orientation during an evaporation process. Therefore, the light out-coupling material has a high refractive index. At last, the light out-coupling material of the target compound is applied to a light out-coupling layer of the electroluminescent device, thereby achieving high efficiency. Moreover, a thickness of the light out-coupling layer of the electroluminescent device is reduced from 85 nm to 65 nm, which effectively saves time and capital costs.

### DESCRIPTION OF THE DRAWINGS

In order to illustrate technical solutions in the embodiments of the present disclosure more clearly, the accompanying drawings required in the description of the embodiments are introduced briefly hereafter. It is obvious that the accompanying drawings in the following description are merely part of the embodiments of the present disclosure. People with ordinary skills in the art can obtain other drawings without making inventive efforts.

FIG. 1 is spectrum graph of a refractive index of a light out-coupling material manufactured by a manufacturing method of an embodiment of the present disclosure.

FIG. 2 is a structural view of an electroluminescent device of an embodiment of the present disclosure.

### REFERENCE NUMERALS IN DRAWINGS

an electroluminescent device 10;
a first electrode 11; a light-emitting functional layer 12; a second electrode 13;
a light out-coupling layer 14; a hole injection layer 121; a hole transport layer 122;
an electron blocking layer 123; a light-emitting layer 124; a hole blocking layer 125;
an electron transport layer 126; and an electron injection layer 127.

### DETAILED DESCRIPTION OF PREFERRED EMBODIMENTS

In the following detailed description, reference is made to the accompanying figures, in which various examples are shown by way of illustration. In this regard, directional terminology mentioned in the present disclosure, such as “top”, “bottom”, “front”, “back”, “left”, “right”, “inner”, “outer”, “lateral”, etc., is used with reference to the orientation of the figures being described. The terminology of the elements mentioned in the present disclosure, such as first, second, etc., are merely used for only distinguishing between different elements and for better expression. In the accompanying figures, units with similar structures are indicated by the same reference numbers.

Embodiments of the present disclosure will be described in detail herein with reference to the drawings. The present disclosure may be embodied in many different forms and the present disclosure is not intended to be construed as being limited to the specific embodiments set forth herein. The embodiments of the present disclosure are provided to explain the practical applications of the present disclosure so that those skilled in the art can understand various embodiments of the present disclosure and various modifications suitable for particular intended applications.

The present disclosure provides a light out-coupling material having the following structural formula:

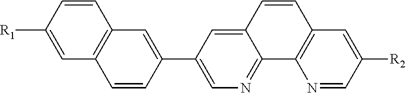

In the structural formula, a group R₁ and a group R₂ each comprises one of an alkyl group, an alkoxy group, or an aromatic group.

The group R₁ comprises one of the following structural formulas:

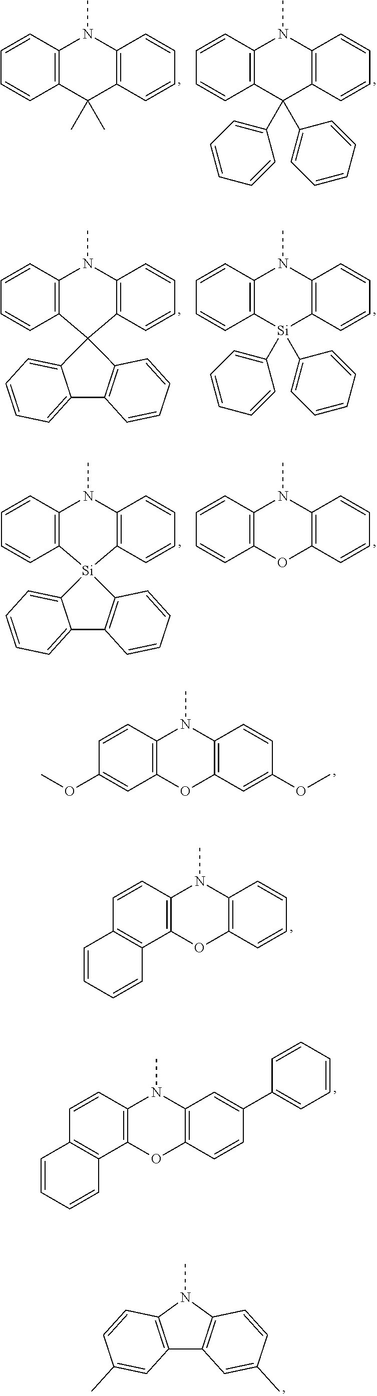

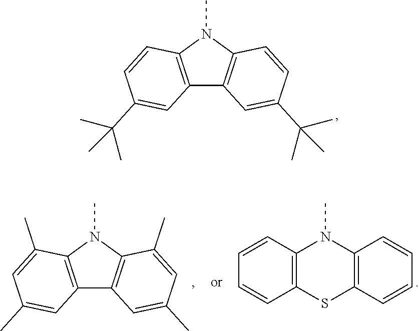

The group R₂ comprises one of the following structural formulas:

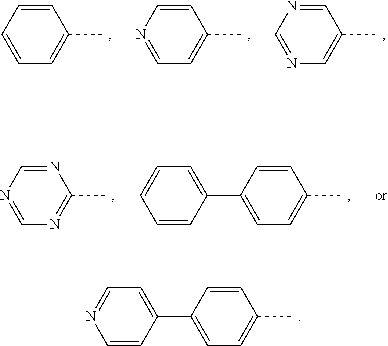

In order to explain the present disclosure more clearly, the light out-coupling optical material will be further explained below in conjunction with the manufacturing method of the light out-coupling material of the present disclosure.

In embodiment I of the present disclosure, a manufacturing method of the light out-coupling material of the present disclosure is described in detail using a manufacturing method of a target compound I (a light out-coupling material of the present disclosure) as an example. A structural formula of the target compound is as follows:

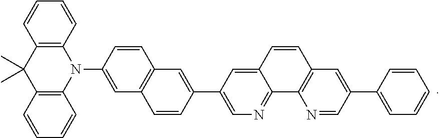 [SMILES: CC1(C)c2ccccc2N(c2ccc3cc(-c4cnc5c(ccc6cc(-c7ccccc7)cnc65)c4)ccc3c2)c2ccccc21]

The manufacturing method of the light out-coupling material of embodiment I comprises the following steps:

Preparing an intermediate having the group R₂, a naphthalene, and a phenanthroline. In the step of preparing the intermediate, specifically comprising the following steps: adding 3-(6-Bromonaphthalen-2-yl)-8-iodo-1,10-phenanthroline, phenylboronic acid, and a catalyst into a Schlenk bottle, and introducing argon gas into the Schlenk bottle; adding deoxygenated toluene, deoxygenated ethanol, and deoxygenated water into the Schlenk bottle, heating the Schlenk bottle under argon protection, and undergoing a reaction at a temperature ranging from 70° C. to 90° C. for 24 h to obtain a second mixture solution; extracting the second mixed solution with dichloromethane several times to obtain a first extract solution; and drying the first extract solution with anhydrous sodium sulfate, filtering and spin-drying the first extract solution, then subjecting the first extract solution to column chromatography using 200 to 300 mesh of silica gel, and being rinsed with an eluent to obtain the intermediate.

A structural formula of the intermediate is as follows:

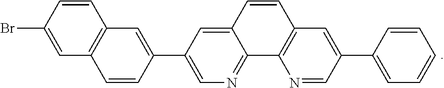 [SMILES: Brc1ccc2cc(-c3cnc4c(ccc5cc(-c6ccccc6)cnc54)c3)ccc2c1]

Adding the intermediate and a first raw material having the group R₁, a catalyst, and sodium t-butoxide into a three-neck flask, and performing gas evacuation and refilling by argon gas. The first raw material is 9,10-dihydro-9,9-dimethylacridine. A molar ratio of the first raw material to the intermediate is 5:8 to 5:6. The catalyst comprises palladium acetate and tri-tert-butylphosphine tetrafluoroborate. A molar ratio of the palladium acetate to the tri-tert-butylphosphine tetrafluoroborate is 1:5 to 1:3.

Adding anhydrous toluene into the reaction vessel to undergo a reaction at a temperature ranging from 110° C. to 130° C. for 24 h, and cooling to room temperature to obtain a first mixed solution.

Introducing the first mixed solution into 180 to 220 ml of ice water, and extracting with dichloromethane several times to obtain an extract solution.

Drying the extract solution with anhydrous sodium sulfate, filtering and spin-drying the extract solution, then subjecting the extract solution to column chromatography using 200 to 300 mesh of silica gel, and being rinsed with an eluent to obtain the target compound I, that is, a light out-coupling material of the present disclosure at a yield of 88%.

In embodiment II of the present disclosure, a manufacturing method of the light out-coupling material of the present disclosure will be described in detail using a manufacturing method of a target compound II (a light out-coupling material of the present disclosure) as an example. A structural formula of the target compound is as follows:

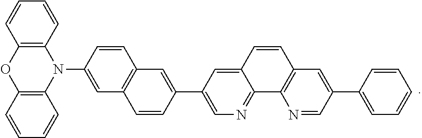 [SMILES: c1ccc(-c2cnc3c(ccc4cc(-c5ccc6cc(N7c8ccccc8Oc8ccccc87)ccc6c5)cnc43)c2)cc1]

The manufacturing method of a light out-coupling material of embodiment II comprises the following steps:

Preparing an intermediate having the group R₂, a naphthalene, and a phenanthroline. The steps of preparing the intermediate is same as the steps of preparing the intermediate of embodiment I.

Adding the intermediate and a first raw material having the group R₁, a catalyst, and sodium t-butoxide into a three-neck flask, and performing gas evacuation and refilling by argon gas. The first raw material is phenothiazine. A molar ratio of the first raw material to the intermediate is 5:8 to 5:6. The catalyst comprises palladium acetate and tri-tert-butylphosphine tetrafluoroborate. A molar ratio of the palladium acetate to the tri-tert-butylphosphine tetrafluoroborate is 1:5 to 1:3.

Adding anhydrous toluene into the reaction vessel to undergo a reaction at a temperature ranging from 110° C. to 130° C. for 24 h, and cooling to room temperature to obtain a first mixed solution.

Introducing the first mixed solution into 180 to 220 ml of ice water, and extracting with dichloromethane several times to obtain an extract solution.

Drying the extract solution with anhydrous sodium sulfate, filtering and spin-drying the extract solution, then subjecting the extract solution to column chromatography using 200 to 300 mesh of silica gel, and being rinsed with an eluent to obtain the target compound II, that is, a light out-coupling material of the present disclosure at a yield of 81%.

In embodiment III of the present disclosure, a manufacturing method of the light out-coupling material of the present disclosure will be described in detail using a manufacturing method of a target compound III (a light out-coupling material of the present disclosure) as an example. A structural formula of the target compound is as follows:

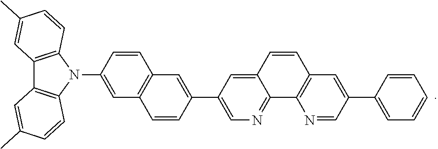 [SMILES: Cc1ccc2c(c1)c1cc(C)ccc1n2-c1ccc2cc(-c3cnc4c(ccc5cc(-c6ccccc6)cnc54)c3)ccc2c1]

The manufacturing method of a light out-coupling material of embodiment III comprises the following steps:

Preparing an intermediate having the group R₂, a naphthalene, and a phenanthroline. The steps of preparing the intermediate is same as the steps of preparing the intermediate of embodiment I.

Adding the intermediate and a first raw material having the group R₁, a catalyst, and sodium t-butoxide into a three-neck flask, and performing gas evacuation and refilling by argon gas. The first raw material is 3,6-dimethylcarbazole. A molar ratio of the first raw material to the intermediate is 5:8 to 5:6. The catalyst comprises palladium acetate and tri-tert-butylphosphine tetrafluoroborate. A molar ratio of the palladium acetate to the tri-tert-butylphosphine tetrafluoroborate is 1:5 to 1:3.

Adding anhydrous toluene into the reaction vessel to undergo a reaction at a temperature ranging from 110° C. to 130° C. for 24 h, and cooling to room temperature to obtain a first mixed solution.

Introducing the first mixed solution into 180 to 220 ml of ice water, and extracting with dichloromethane several times to obtain an extract solution.

Drying the extract solution with anhydrous sodium sulfate, filtering and spin-drying the extract solution, then subjecting the extract solution to column chromatography using 200 to 300 mesh of silica gel, and being rinsed with an eluent to obtain the target compound III, that is, a light out-coupling material of the present disclosure at a yield of 75%.

The light out-coupling material are prepared by the manufacturing method of the embodiments of the present disclosure. Therefore, the light out-coupling material can be effectively synthesized and the synthesis efficiency can be increased at the same time.

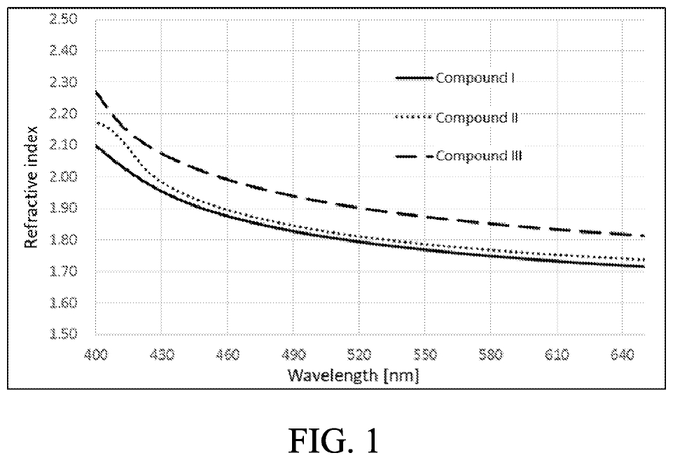

In order to verify whether characteristics of the light out-coupling material of the present disclosure satisfy requirements of the electroluminescent device, the light out-coupling material obtained by the manufacturing method of the present embodiment is subjected to a determination of photophysical properties. A graph of a refractive index versus wavelengths was obtained, as shown in FIG. 1.

As shown in FIG. 1, at a wavelength of 400 nm, a refractive index of the target compound III of the present disclosure is greater than a refractive index of the target compound II. The refractive index of the target compound II is greater than a refractive index of the target compound I. The refractive index of the target compound I, the target compound II and the target compound III decreases as the wavelength increases.

Therefore, the light out-coupling material manufactured by the present disclosure can obtain light out-coupling material having a high refractive index by controlling wavelengths.

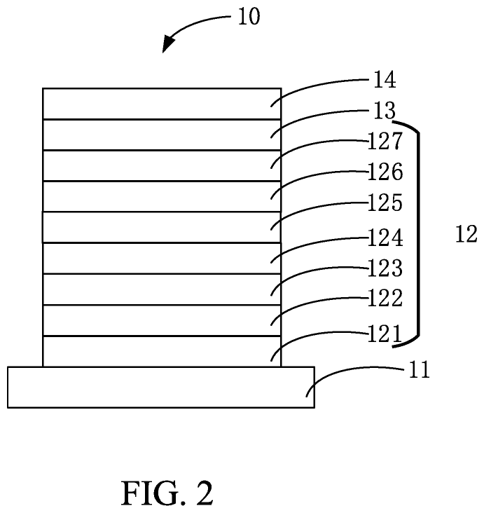

As shown in FIG. 2, the present disclosure also provides an electroluminescent device 10 comprising the light out-coupling material.

Specifically, the electroluminescent device includes a first electrode 11, a light-emitting functional layer 12, a second electrode 13, and a light out-coupling layer 14.

The first electrode 11 is an anode. The light-emitting functional layer 12 is disposed on the first electrode 11. The second electrode 13 is disposed on the light-emitting functional layer 12. The second electrode 13 is a cathode. The light out-coupling layer 14 is disposed on the second electrode 13. A material used for the light out-coupling layer 14 includes the light out-coupling material.

The light-emitting functional layer 12 includes a hole injection layer 121, a hole transport layer 122, an electron blocking layer 123, a light-emitting layer 124, a hole blocking layer 125, an electron transport layer 126, and an electron injection layer 127.

The hole injection layer 121 is disposed on the first electrode 11. The hole transport layer 122 is disposed on a side of the first electrode 11 of the hole injection layer 121. The electron blocking layer 123 is disposed a side of the hole transport layer 122 away from the hole injection layer 121. The light-emitting layer 124 is disposed on a side of the electron blocking layer 123 away from the hole transport layer 122. The layer 125 is disposed on a side of the light-emitting layer 124 away from the electron blocking layer 123. The electron transport layer 126 is disposed on a side of the hole blocking layer 125 away from the light-emitting layer 124. The electron injecting layer 127 is disposed on a side of the electron transport layer 126 away from the hole blocking layer 123.

Table 1 is a performance data table of the electroluminescent device 10 that employs the target compound I, the target compound II, or the target compound III.

| | | | | Maximum |
|---|---|---|---|---|
| | | Maximum | | external |
| | | current | | quantum |
| | CPL | efficiency | | efficiency |
| Device | Material | (cd/A) | (CIEx, CIEy) | (%) |
| Device 1 | Compound 1 | 6.6 | (0.13, 0.046) | 14.3% |
| Device 2 | Compound 2 | 7.1 | (0.13, 0.045) | 15.1% |
| Device 3 | Compound 3 | 7.4 | (0.13, 0.045) | 15.4% |

In the electroluminescent device 10 of the present disclosure, the light out-coupling layer 14 employs the photo-coupled output material to effectively manufacture the electroluminescent device 10, thereby increasing luminous efficiency of the electroluminescent device. Furthermore, a thickness of the light out-coupling layer 14 is reduced from 85 nm to 65 nm, which saves time and capital costs.

Technical scopes of the present disclosure are not limited to contents of the description. People skilled in the art may further make modifications and improvements without departing from the principle of the present disclosure, and these modifications and improvements shall also fall within the scope of the present disclosure.

1. A light out-coupling material comprising the following general structural formula: 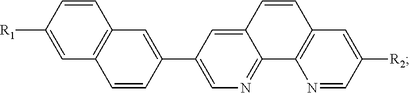
wherein a group R₁ and a group R₂ each comprises one of an alkyl group, an alkoxy group, or an aromatic group.

2. The optical out-coupling material according to claim 1, wherein the group R₁ comprises one of the following structural formulas:

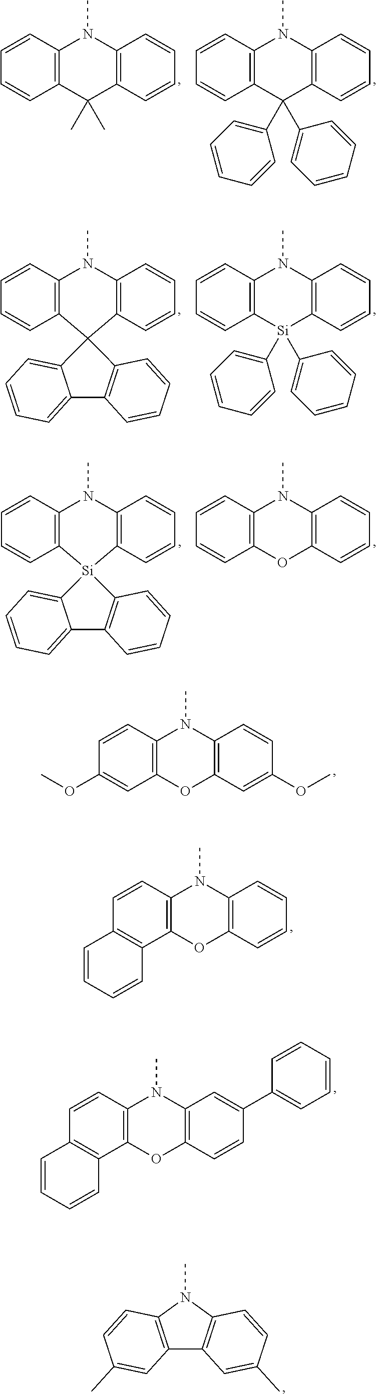

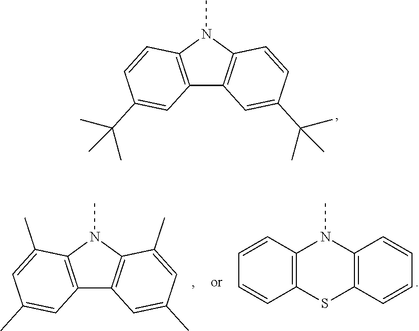

3. The light out-coupling material according to claim 1, wherein the group R₂ comprises one of the following structural formulas:

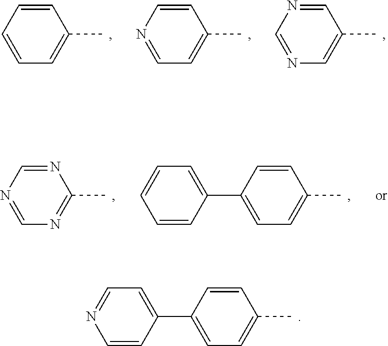

4. A manufacturing method of the light out-coupling material according to claim 1, wherein the manufacturing method comprises the following steps:
preparing an intermediate having the group R₂, a naphthalene, and a phenanthroline;
adding the intermediate and a first raw material having the group R₁, a catalyst, and sodium t-butoxide into a three-neck flask, and performing gas evacuation and refilling by argon gas;
adding anhydrous toluene into the three-neck flask to undergo a reaction at a temperature ranging from 110° C. to 130° C. for 24 h, and cooling to room temperature to obtain a first mixed solution;
introducing the first mixed solution into 180 to 220 ml of ice water, and extracting with dichloromethane several times to obtain an extract solution; and
drying the extract solution with anhydrous sodium sulfate, filtering and spin-drying the extract solution, then subjecting the extract solution to column chromatography using 200 to 300 mesh of silica gel, and being rinsed with an eluent to obtain the light out-coupling material.

5. The manufacturing method according to claim 4, wherein the step of preparing the intermediate specifically comprises the following steps:
adding 3-(6-Bromonaphthalen-2-yl)-8-iodo-1,10-phenanthroline, phenylboronic acid, and a catalyst into a Schlenk bottle, and introducing argon gas into the Schlenk bottle;
adding deoxygenated toluene, deoxygenated ethanol, and deoxygenated water into the Schlenk bottle, heating the Schlenk bottle under argon protection, and undergoing a reaction at a temperature ranging from 70° C. to 90° C. for 24 h to obtain a second mixture solution;
extracting the second mixed solution with dichloromethane several times to obtain a first extract solution; and
drying the first extract solution with anhydrous sodium sulfate, filtering and spin-drying the first extract solution, then subjecting the first extract solution to column chromatography using 200 to 300 mesh of silica gel, and being rinsed with an eluent to obtain the intermediate.

6. The manufacturing method according to claim 4,
wherein the first raw material comprises phenothiazine, 9,10-dihydro-9,9-dimethylacridine, and 3,6-dimethylcarbazole;
a molar ratio of the first raw material to the intermediate is 5:8 to 5:6;
the catalyst comprises palladium acetate and tri-tert-butylphosphine tetrafluoroborate; and
a molar ratio of the palladium acetate to the tri-tert-butylphosphine tetrafluoroborate is 1:5 to 1:3.

7. The manufacturing method according to claim 4,
wherein a molar ratio of the 3-(6-bromonaphthalen-2-yl)-8-iodo-1,10-phenanthroline to the phenylboronic acid is from 10:9 to 10:5.

8. An electroluminescent device comprising the light out-coupling material of claim 1.

9. The electroluminescent device according to claim 8, wherein the electroluminescent device comprises:
a first electrode;
a light-emitting functional layer disposed on the first electrode;
a second electrode disposed on the light-emitting functional layer; and
a light out-coupling layer disposed on the second electrode, wherein a material used for the light out-coupling layer comprises the light out-coupling material.

10. The electroluminescent device according to claim 9, wherein the luminescent functional layer comprises:
a hole injection layer disposed on the first electrode;
a hole transport layer disposed on a side of the first electrode of the hole injection layer;
an electron blocking layer disposed on a side of the hole transport layer that is away from the hole injection layer;
a light-emitting layer disposed on a side of the electron blocking layer that is away from the hole transport layer;
a hole blocking layer disposed on a side of the light-emitting layer that is away from the electron blocking layer;
an electron transport layer disposed on a side of the hole blocking layer that is away from the light-emitting layer; and
an electron injection layer disposed on a side of the electron transport layer that is away from the hole blocking layer.
